(19) United States
(12) Patent Application Publication
(10) Pub. No.: US 2026/0311817 A1
(43) Pub. Date: Oct. 8, 2026

(54) DRUG MOLECULE TARGETED GENERATION METHOD AND SYSTEM

(71) Applicant: DEEPMED TECHNOLOGY (SUZHOU) CO., LTD., Suzhou (CN)

(72) Inventors: Yang ZHANG, Suzhou (CN); Lin WANG, Suzhou (CN); Jin LIU, Suzhou (CN); Jun ZHANG, Suzhou (CN)

(21) Appl. No.: 19/442,898

(22) Filed: Jan. 7, 2026

(30) Foreign Application Priority Data
Feb. 26, 2025 (CN) 202510220294.7

(51) Int. Cl. G16C 20/50; G16C 20/30; G16C 20/70; G16C 20/80

(52) U.S. Cl. CPC G16C 20/50; G16C 20/30; G16C 20/70; G16C 20/80

(57) ABSTRACT

The invention provides a method and system for targeted generation of drug molecules. The method includes: constructing a molecular representation base model to perform transformation, updating, pooling, and integration of chemical space information of drug molecular structures, generating a one-dimensional molecular encoding vector that reflects molecular structure and pharmacodynamic property information; constructing a molecular structure generation model and a molecular property prediction model, and performing segmented training based on the one-dimensional molecular encoding vector. The former restores the vector to a drug molecular structural formula through dimension expansion, feature transformation, and decoding, while the latter predicts molecular attributes. Based on the molecular property prediction results, targeted optimization and generation of new drug molecular structures with desired pharmacodynamic properties can be achieved. By accurately modeling the distribution patterns of drug chemical space, the invention effectively improves the efficiency and success rate of drug research and development.

### FIELD OF THE INVENTION

The present invention relates to the technical field of drug molecule design, and specifically discloses a method and system for targeted generation of drug molecules.

### DESCRIPTION OF THE RELATED ART

In the field of drug research and development, efficiently and accurately designing drug molecules with specific structures and properties has always been a goal pursued by researchers. With the continuous development of computer technology and machine learning algorithms, an increasing number of molecular design methods have been proposed to assist the drug research and development process.

In early molecular design methods, REINVENT served as a molecular design framework based on reinforcement learning and SMILES generation. It optimized the structure of input molecules using reinforcement learning, with the molecular SMILES expression as the core optimization object, thereby generating new molecules. However, this method has significant drawbacks: its optimization object is a complete, independent molecular structure, which prevents continuous targeted optimization in the encoding space, resulting in low search efficiency. When facing large-scale molecular search tasks, it is difficult to quickly find molecular structures that meet specific requirements.

LatentGAN and MDM, two molecular generation frameworks, also have certain applications in molecular design. They primarily achieve molecular generation by constructing latent space vectors. However, these latent space vectors are designed solely for molecular generation tasks and lack effective representation of molecular pharmacodynamic characteristics. This means that in the drug design process, relying solely on molecules generated by these methods makes it difficult to ensure good pharmacodynamic effects, failing to meet the practical requirements of drug research and development for molecular functionality.

ScaffoldGVAE is also a molecular generation framework, but its entire molecular generation process is fragmented into multiple steps. This necessitates reliance on specific input molecular combinations to constrain the direction of molecular generation, thus preventing targeted molecular optimization. In practical applications, when designing molecules based on specific drug targets or pharmacodynamic requirements, this limitation of ScaffoldGVAE makes it difficult to flexibly and accurately generate molecules that meet the requirements.

RegressionTransformer, as a conditional molecular generation framework, also has its characteristics in molecular design. However, this method requires explicit modeling of molecular property information within the generation model, leading to poor generalization and universality of the model. In specific tasks of drug design, especially with small datasets, fine-tuning the model often fails to achieve ideal results, limiting its application in practical drug research and development.

In summary, existing molecular design methods have numerous shortcomings in molecular structure optimization, pharmacodynamic characteristic representation, targeted molecular generation, and model generalization. Therefore, there is an urgent need for a new molecular design technical solution that can effectively address the above issues, improve the efficiency and accuracy of molecular design, and provide stronger support for drug research and development.

### SUMMARY OF THE INVENTION

To resolve the above technical problems, the present invention provides a drug molecule targeted generation method and system. The method comprises the following steps:
step S1: acquiring a drug molecular structure, to construct a molecular representation base model; based on the molecular representation base model, performing information transformation, information updating, information pooling, and information integration operations on the chemical space information of the drug molecular structure, generating a one-dimensional molecular encoding vector capable of representing molecular structure information and pharmacodynamic property information, and optimizing the molecular representation base model based on intermolecular similarity in the one-dimensional molecular encoding vector;
step S2: constructing a molecular structure generation model and a molecular property prediction model, performing segmented training on the molecular structure generation model and the molecular property prediction model based on the one-dimensional molecular encoding vector, obtaining a trained molecular structure generation model and a trained molecular property prediction model, including:
   restoring the one-dimensional molecular encoding vector to a drug molecular structural formula via the trained molecular structure generation model through dimension expansion, feature transformation, and decoding, to ensure a one-to-one correspondence between the one-dimensional molecular encoding vector and its drug molecular structural formula; and
   based on the one-dimensional molecular encoding vector, obtaining one or more molecular property prediction results via the trained molecular property prediction model; and
step S3: based on the molecular property prediction results of the one-dimensional molecular encoding vector, optimizing the one-dimensional molecular encoding vector via backpropagation, and restoring the optimized one-dimensional molecular encoding vector to a molecular structure, to achieve targeted optimization of a drug molecule.

In one embodiment of the present invention, in step S1, the method for generating the one-dimensional molecular encoding vector capable of representing molecular structure information and pharmacodynamic property information is as follows:
S11: converting the drug molecular structure into a SMILES string, and standardizing the format of the SMILES string to obtain a preprocessed SMILES string;
S12: converting the preprocessed SMILES string into a molecular graph, and based on atomic and chemical bond markers in the molecular graph, converting the molecular graph into a homogeneous graph composed of nodes representing atoms and edges representing chemical bonds;
S13: updating the node representations and edge representations in the homogeneous graph to obtain an updated molecular graph representation;
S14: inputting each node representation from the updated molecular graph representation into a multilayer perceptron to predict the attention weight of each node, and inputting the attention weights into a SoftMax activation function for normalization to obtain a normalized attention weight;
S15: multiplying the normalized attention weights with the corresponding node representations, followed by sum pooling, to obtain the one-dimensional molecular encoding vector.

In one embodiment of the present invention, in step S13, the method for updating the node representations and edge representations in the homogeneous graph is as follows:
S131: for the initial feature representation of each node, projecting the initial feature representation to a higher-dimensional space via linear transformation to obtain input nodes;
S132: for each edge connecting any two input nodes, iteratively updating the current edge feature by concatenating the features of any node and the edge, to obtain updated edges;
S133: based on the updated edges, for each node, aggregating the features of all connected edges and fusing the aggregation result with the feature of the node to iteratively update the current node feature, to obtain a currently updated node; and
S134: for any adjacent node of the currently updated node, performing element-wise multiplication of the feature of the adjacent node and the feature of the updated edge to obtain the integrated feature of the adjacent node, thereby calculating the integrated features of all adjacent nodes of the updated node and aggregating them, and performing element-wise multiplication of the aggregated features of the adjacent node with features generated by a self-loop attention mechanism to obtain the final updated node features.

In one embodiment of the present invention, the method for updating the node representations and edge representations in the homogeneous graph further includes: introducing information such as molecular 3D conformation, conformational dynamics, conformational space, pharmacophore features, and molecular shape to update the feature of each node and the feature of each edge in the homogeneous graph.

In one embodiment of the present invention, in step S1, the method for optimizing the molecular representation base model based on intermolecular similarity in the one-dimensional molecular encoding vector is as follows:
using a multilayer perceptron as a projection head, inputting any two sets of one-dimensional molecular encoding vectors after concatenation and shape adjustment into the projection head to obtain a predicted similarity matrix between the two sets of molecular encoding vectors;
calculating the similarity between different molecules in the drug molecular structure to obtain a true similarity matrix; and
calculating the error between the predicted similarity matrix and the true similarity matrix, and adjusting the parameters of the molecular representation base model via backpropagation based on the error.

In one embodiment of the present invention, any element in the true similarity matrix represents one or more similarities between different molecules.

In one embodiment of the present invention, the method for obtaining the trained molecular structure generation model is as follows:
performing autoregressive decoding on the one-dimensional molecular encoding vector based on the molecular structure generation model, calculating the predicted probability of each character representing different molecules at the current position, and obtaining a predicted vocabulary based on the predicted probabilities;
segmenting the SMILES string converted from the drug molecular structure into multiple character tokens, and summarizing all character tokens based on their actual position information in the original SMILES string to form a true vocabulary; and
calculating the character error between corresponding positions in the predicted vocabulary and the true vocabulary, and adjusting the parameters of the molecular structure generation model via backpropagation based on the character error until the character error falls within a preset range, to obtain the trained molecular structure generation model.

In one embodiment of the present invention, in step S2, the method for restoring the one-dimensional molecular encoding vector to a drug molecular structural formula via the trained molecular structure generation model is as follows:
using the one-dimensional molecular encoding vector as a memory vector, performing autoregressive decoding on the memory vector via a Transformer decoder structure to obtain a feature vector sequence;
performing character decoding on the feature vector sequence via a multilayer perceptron to obtain predicted scores for each character;
based on the predicted scores, calculating the probability distribution value of each character appearing at the current position, and predicting the output character at the current position based on the probability distribution value, after generating the first character, mapping the first character to a low-dimensional embedding vector and concatenating it with previous feature vectors, then inputting the concatenated vector into the multilayer perceptron again to predict the next character, repeating this prediction process until the generated string meets a specified length, ultimately obtaining a SMILES string composed of multiple characters; and
restoring the SMILES string to a drug molecular structural formula.

In one embodiment of the present invention, the method for obtaining one or more molecular property prediction results via the trained molecular property prediction model is: transforming the one-dimensional molecular encoding vector into molecular property prediction values via a deep neural network after multiple layers of dimension expansion, batch normalization, and activation functions.

Based on the same inventive concept, the present invention also provides a drug molecule targeted generation system configured to implement the aforementioned drug molecule targeted generation method. The drug molecule targeted generation system includes the following modules:
a molecular representation module, configured to acquire drug molecular structures, to construct a molecular representation base model, and based on the molecular representation base model, perform information transformation, information updating, information pooling, and information integration operations on the chemical space information of said drug molecular structures, generate a one-dimensional molecular encoding vector capable of representing molecular structure information and pharmacodynamic property information, and optimize the molecular representation base model based on intermolecular similarity in the one-dimensional molecular encoding vector;
a decoding module, configured to construct a molecular structure generation model and a molecular property prediction model, perform segmented training on the molecular structure generation model and the molecular property prediction model based on the one-dimensional molecular encoding vector, obtain a trained molecular structure generation model and a trained molecular property prediction model, and respectively obtain molecular structural formulas and molecular property prediction results via the trained molecular structure generation model and the trained molecular property prediction model; and a molecular targeted optimization module, configured to optimize the one-dimensional molecular encoding vector via backpropagation based on the molecular property prediction results of the one-dimensional molecular encoding vector, and restore the optimized one-dimensional molecular encoding vector to a molecular structure, to achieve targeted optimization of drug molecules;
Wherein, the decoding module includes a molecular structure generation submodule and a molecular property prediction submodule, the molecular structure generation submodule restores the one-dimensional molecular encoding vector to a molecular structural formula via the trained molecular structure generation model; the molecular property prediction submodule obtains one or more molecular property prediction results based on the one-dimensional molecular encoding vector via the trained molecular property prediction model. The above technical solution of the present invention has the following advantages over the prior art:

First, information integration and efficient representation: By constructing a molecular representation base model, the chemical space information of drug molecular structures can be comprehensively transformed, updated, pooled, and integrated, generating a one-dimensional molecular encoding vector. This approach effectively integrates molecular structure information and pharmacodynamic property information, efficiently representing key features of drug molecules in a concise one-dimensional vector form, facilitating subsequent model processing, and improving computational efficiency and information transmission efficiency.

Second, multi-task processing based on the one-dimensional encoding vector obtained from the molecular representation base model can restore molecular structural formulas or predict multiple molecular attributes. This multi-task capability not only helps comprehensively understand drug molecular characteristics but also provides a rich data foundation for drug research and development. Based on the restored molecular structural formulas and property prediction results, targeted optimization and generation of new drug molecular structures with desired pharmacodynamic properties can be achieved, expanding the application scope of the technology in the field of drug research and development, and improving research and development efficiency and success rate.

### BRIEF DESCRIPTION OF THE DRAWINGS

To make the content of the present invention more easily understood clearly, the present invention is further described in detail below with reference to specific embodiments and accompanying drawings, wherein:

FIG. 1 is a schematic flowchart of a drug molecule targeted generation method in an embodiment of the present invention;

FIG. 2 is a schematic flowchart of a method for generating a one-dimensional molecular encoding vector capable of representing molecular structure information and pharmacodynamic property information in an embodiment of the present invention;

FIG. 3 is a schematic structural diagram of a molecular representation base model in an embodiment of the present invention;

FIG. 4 is a schematic structural diagram of a structural decoder for molecular representation vectors obtained in an embodiment of the present invention;

FIG. 5 is a performance comparison in similarity-based virtual screening using the molecular representation method of the present invention;

FIG. 6 is a test of the molecular structure decoder's ability to generate new molecules by increasing temperature and adding noise;

FIG. 7 is the performance of the Ouroboros encoder combined with a multilayer perceptron in molecular property prediction in the present invention;

FIG. 8 shows the migration results of two PDE4B inhibitors in the Ouroboros chemical encoding space of the present invention;

FIG. 9 is a schematic structural diagram of a drug molecule targeted generation system in an embodiment of the present invention;

Reference numbers: 100: molecular representation module; 200: decoding module; 200a: molecular structure generation submodule; 200b: molecular property prediction submodule; 300: molecular targeted optimization module.

### DETAILED DESCRIPTION OF THE EMBODIMENTS

The specific embodiments of the present invention are further described below with reference to the accompanying drawings to enable those skilled in the art to better understand the present invention and implement it, but the examples given are not intended to limit the present invention.

### Embodiment 1

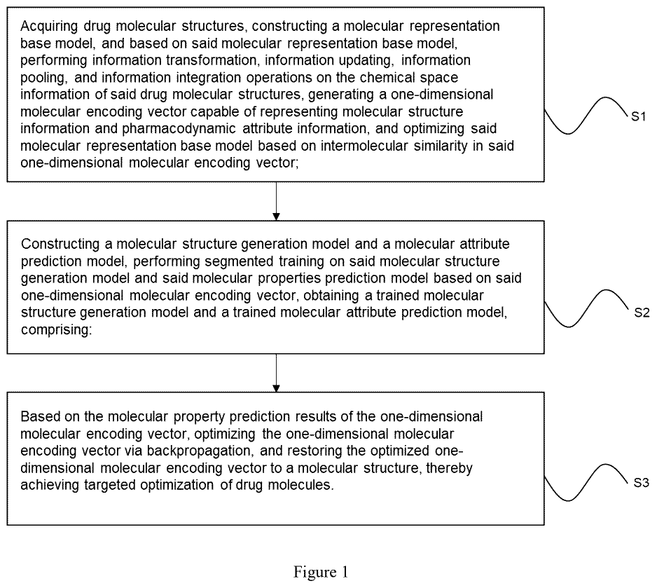

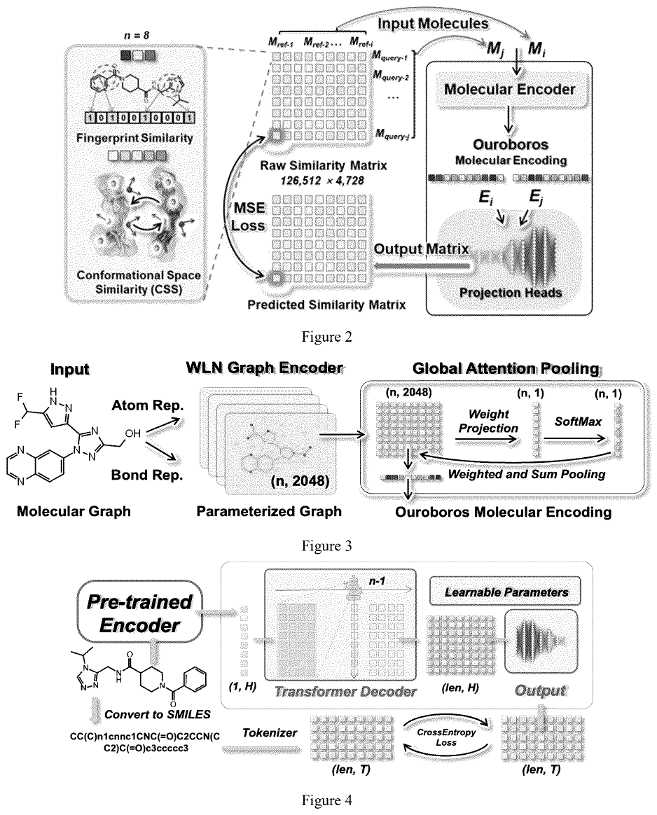

Referring to FIG. 1, the present invention, based on the Ouroboros basic architecture, provides a drug molecule targeted generation method. The method includes the following steps:
Step S1: acquiring drug molecular structures, to construct a molecular representation base model, and based on the molecular representation base model, performing information transformation, information updating, information pooling, and information integration operations on the chemical space information of the drug molecular structures, generating a one-dimensional molecular encoding vector capable of representing molecular structure information and pharmacodynamic property information;
Step S2: constructing a molecular structure generation model and a molecular property prediction model, performing segmented training on the molecular structure generation model and the molecular property prediction model based on the one-dimensional molecular encoding vector, obtaining a trained molecular structure generation model and a trained molecular property prediction model, including: restoring the one-dimensional molecular encoding vector to a drug molecular structural formula via the trained molecular structure generation model through dimension expansion, feature transformation, and decoding, to ensure a one-to-one correspondence between the one-dimensional molecular encoding vector and its drug molecular structural formula; and based on the one-dimensional molecular encoding vector, obtaining one or more molecular property prediction results via the trained molecular property prediction model;
Step S3: based on the molecular property prediction results of the one-dimensional molecular encoding vector, optimizing the one-dimensional molecular encoding vector via backpropagation, and restoring the optimized one-dimensional molecular encoding vector to a molecular structure, to achieve targeted optimization of drug molecules.

Further, the main architecture of the molecular representation base model includes: transformation of drug molecular structures, information updating, information pooling, and information integration. Its implementation methods include chemical language models, molecular graph models, etc.

Taking the molecular graph model as an example, the molecular representation base model includes generation of molecular graphs, graph neural networks for extracting molecular information, pooling networks for compressing updated graph data into one dimension, and information integration networks.

In step S1, as shown in FIG. 3, based on the molecular representation base model, the method for performing information transformation, information updating, information pooling, and information integration operations on the chemical space information of the drug molecular structures, generating a one-dimensional molecular encoding vector capable of representing molecular structure information and pharmacodynamic property information, is as follows:
S11: converting the drug molecular structure into a SMILES string, and standardizing the format of the SMILES string to obtain a preprocessed SMILES string;
S12: Using a byte pair tokenizer to segment the preprocessed SMILES string into multiple tokens, each token representing a single atom (e.g., “C” for carbon) or chemical bond (e.g., “=” for double bond) or substructure fragment (e.g., “Cl” for chlorine atom), assigning an identifier to each token, converting all tokens into a molecular graph, and based on atomic and chemical bond markers in the molecular graph, converting it into a homogeneous graph G=(V,E), where V represents the set of atoms and E represents the set of chemical bonds;
S13: using a graph neural network (GNN) Weisfeiler-Lehman Network (WLN) to update the node representations and edge representations in the homogeneous graph, obtaining an updated molecular graph representation; the node representations include but are not limited to atom type, atom hybridization, atom charge, atom chirality, whether located in a ring, whether aromatic; the edge representations include but are not limited to chemical bond order, whether part of a conjugated system, whether located in a ring;
S14: using a global self-attention pooling module, inputting each node representation hᵢ from the updated molecular graph representation into a multilayer perceptron (MLP) to predict the attention weight aᵢ of each node. To ensure the sum of all attention weights equals 1, inputting the attention weights aᵢ into a Softmax activation function for normalization to obtain normalized attention weights wᵢ:

$${w}_{i} = \frac{exp ( {a}_{i} )}{{\sum }_{i} exp ( {a}_{i} )}$$

S15: multiplying the normalized attention weights wᵢ with the corresponding node representations hᵢ, followed by sum pooling, to generate a graph-level node encoding H_{graph}, i.e., the one-dimensional molecular encoding vector:

$${H}_{graph} = {\sum }_{\{ i ϵ V \}} {h}_{i} \cdot {w}_{i} .$$

Further, in the above step S13, the method for updating the node (atom) representations and edge (chemical bond) representations in the homogeneous graph via the graph neural network (GNN) Weisfeiler-Lehman Network (WLN) using a message-passing approach is as follows:
S131: Node feature projection: for the initial feature representation hᵥ of each atom, projecting the initial atom feature hᵥ to a higher-dimensional space via linear transformation, then using a ReLU activation function to obtain the atom feature

$${h}_{v}^{( 0 )}$$

input to the WLN:

$${h}_{v}^{( 0 )} = R e L U ( {W}_{i n} {h}_{v} + {b}_{i n} )$$

where Wᵢₙ represents a trainable weight matrix, bᵢₙ is a bias parameter.
S132: edge feature update: for each chemical bond connecting atom u and atom v, updating the chemical bond feature

$${e}_{u v}^{( t )}$$

by concatenating the feature hᵤ of atom u and the chemical bond feature

$${e}_{u v}^{( C )} ,$$

to obtain the updated chemical bond feature

$${e}_{u v}^{( t + 1 )} :$$

$${e}_{u v}^{( t + 1 )} = R e L U ( {W}_{contact} [ {h}_{u} \vert \vert {e}_{u v}^{( t )} ] + {b}_{contact} )$$

where t represents the number of message-passing iterations,

$${e}_{u v}^{( t + 1 )}$$

represents the chemical bond feature after (t+1) updates,

$${e}_{u v}^{( t )}$$

represents the chemical bond feature after t updates, ∥ represents the concatenation operation, W_{contact} represents the trainable weight matrix in the concatenation operation, b_{contact} represents the bias parameter in the concatenation operation;
S133: node feature aggregation and update: iteratively updating the current atom feature

$${h}_{v}^{( t )}$$

by aggregating all adjacent bond features for each atom and fusing the aggregation result with the atom feature, to obtain the currently updated atom feature

$${h}_{v}^{( t + 1 )}$$

$${h}_{v}^{( t + 1 )} = ReLU ( {W}_{aggregate} [ {h}_{v}^{( t )} \vert \vert {\sum }_{u ϵ N ( v )} {e}_{u v}^{( t + 1 )} ] + {b}_{aggregate} )$$

where, N(v) is the set of adjacent atoms of atom v, t represents the number of message-passing iterations; W_{aggregate} represents the trainable weight matrix in the aggregation operation; b_{aggregate} represents the bias parameter in the aggregation operation;
S134: attention mechanism and final feature update: for any adjacent atom u of atom v, performing element-wise multiplication of the adjacent atom feature huy and the updated bond feature

$${e}_{u v}^{( t + 1 )}$$

to obtain the integrated feature of the adjacent atom, thereby calculating the integrated features of all adjacent atoms of atom v and aggregating them, and performing element-wise multiplication of the aggregated adjacent atom features with features generated by a self-loop attention mechanism to obtain the final updated atom feature

$${h}_{v}^{final} :$$

$${h}_{v}^{final} = {W}_{self} {h}_{v}^{( t + 1 )} ⊙ ( {\sum }_{u ϵ N ( v )} ( {W}_{b o n d} {e}_{u v}^{( t + 1 )} {W}_{atom} {h}_{u v} ) )$$

where ⊚ represents element-wise product, W_{self} represents the self-loop attention weight matrix, W_{bond} represents the bond feature weight matrix, Wₐₜₒₘ represents the atom feature weight matrix, hᵤᵥ represents the feature representation of neighbor atom u connected to atom v.

Further, the method for optimizing the molecular representation base model based on intermolecular similarity in the one-dimensional molecular encoding vector is as follows: using a multilayer perceptron as a projection head, inputting any two sets of one-dimensional molecular encoding vectors after concatenation and shape adjustment into the projection head to obtain a predicted similarity matrix between the two sets of molecular encoding vectors; calculating the similarity between different molecules in the drug molecular structure to obtain a true similarity matrix; wherein, any element in the true similarity matrix represents one or more similarities between different molecules, including but not limited to molecular fingerprint similarity and conformational space similarity between molecules, wherein molecular fingerprint similarity includes but is not limited to topological molecular fingerprints (AtomPairs), circular fingerprints (ECFP4), substructure fingerprints (MACCS), etc., and conformational space similarity refers to characteristic values (including maximum, average, minimum, etc.) of the similarity distribution between conformational ensembles of molecules under different energy windows;

calculating the error between the predicted similarity matrix and the true similarity matrix via mean squared error loss, and adjusting the parameters of the molecular representation base model via backpropagation based on the error.

For molecular 3D conformation information, the three-dimensional distances and angles between atoms in the molecule can be calculated, these geometric features are encoded into vector form, and concatenated or fused with the word-embedded vectors. For example, distance information between atoms can be added as a new feature dimension to the vector representation of the molecular sequence, allowing the model to learn the structural features of the molecule in three-dimensional space. For pharmacophore features, based on known pharmacophore models, pharmacophore parts in the molecule can be identified and their features integrated into the vector representation of the corresponding tokens, enabling the model to learn information related to pharmacodynamics.

Further, in step S2, the method for obtaining the trained molecular structure generation model with reference to the training process shown in FIG. 4 is as follows:
performing autoregressive decoding on the one-dimensional encoding vector based on the Transformer decoder of the molecular structure generation model, calculating the predicted probability of each character representing different molecules at the current position, and constructing a predicted vocabulary based on the predicted probabilities;
using a tokenizer to segment the SMILES string converted from the drug molecular structure into multiple character tokens, and summarizing all character tokens based on their actual position information in the original SMILES string to form a true vocabulary; and
calculating the error between corresponding character tokens in the predicted vocabulary and the true vocabulary via weighted cross-entropy loss, and adjusting the parameters of the molecular structure generation model via backpropagation based on the character error until the character error falls within a preset range, thereby obtaining the trained molecular structure generation model.

In the inference phase, the method for restoring the one-dimensional molecular encoding vector to a drug molecular structural formula via the above-trained molecular structure generation model is as follows:
using the one-dimensional molecular encoding vector as a memory vector, performing autoregressive decoding on the memory vector via a Transformer decoder structure to obtain a feature vector sequence;
performing character decoding on the feature vector sequence via a multilayer perceptron to obtain predicted scores for each character; wherein, the multilayer perceptron includes multiple fully connected layers, with the number of neurons in the last layer equal to the number of characters in the molecular structural formula; and
based on the predicted scores, calculating the probability distribution value of each character appearing at the current position, and predicting the output character at the current position based on the maximum value of all probability distribution values, or sampling with temperature-related noise. After generating the first character, mapping the character to a low-dimensional embedding vector and concatenating it with previous feature vectors, then inputting the concatenated vector into the multilayer perceptron again to predict the next character. During the generation process, null tokens are ignored, and the generation process ends when a termination token is encountered, ultimately obtaining a chemical language representation composed of a series of tokens, i.e., a complete SMILES string, and restoring the SMILES string to a drug molecular structural formula, thereby achieving the purpose of restoring the one-dimensional molecular encoding vector to a drug molecular structural formula.

Further, before performing molecular property prediction, the drug molecular structure to be predicted needs to be encoded into a one-dimensional molecular encoder vector via the molecular representation base model, and the one-dimensional molecular encoder vector is transformed into a 2048-dimensional molecular encoding vector. This vector contains key information such as the molecular chemical structure and pharmacophore features, providing a data foundation for subsequent property prediction.

The molecular property prediction model consists of three neural network components. The above 2048-dimensional molecular encoding vector is input into the trained molecular property prediction mode.

The first component includes a dropout layer with a dropout rate of 0.5, encoding the drug molecular structure to be predicted into a one-dimensional molecular encoder vector via the molecular representation base model, and transforming the one-dimensional molecular encoder vector into a 2048-dimensional molecular encoding vector. The molecular encoding is projected from 2048 dimensions to an 8192-dimensional feature space. After completing the dimension projection, a sigmoid activation function is applied to the features for nonlinear transformation, thereby introducing nonlinear characteristics and enhancing the model's expressive ability.

The second component concatenates the 8192-dimensional molecular encoding generated by the first component with the original 2048-dimensional molecular encoding to obtain a 10240-dimensional vector. The concatenated 10240-dimensional vector sequentially passes through two linear layers for feature transformation. During the processing of these two linear layers, the feature dimension remains 10240. To further enhance the model's learning ability and stability, LeakyReLU activation functions and batch normalization operations are applied between the two linear layers, respectively.

The third component performs element-wise addition of the input and output features of the second component to achieve feature fusion, obtaining fused features. The fused features first undergo feature transformation via a linear layer, then batch normalization is applied to normalize the features, followed by a SiLU (Sigmoid-weighted Linear Unit) activation function to introduce nonlinearity. Subsequently, the features are projected to a 1024-dimensional feature space, undergoing another round of batch normalization and SiLU activation function processing. Finally, the features are projected to the dimension of the target property via another linear layer with a SiLU activation function. In benchmark test scenarios, all target properties are one-dimensional.

After processing by the molecular property prediction model, the predicted molecular property value is ultimately output. This value represents the model's prediction result for a specific property of the molecule (such as solubility, membrane permeability, lipophilicity, etc.), which can be used to evaluate the potential of the molecule in drug research and development, enable targeted optimization and generation of new drug molecular structures with desired pharmacodynamic attributes, and help researchers screen and design more active drug molecules.

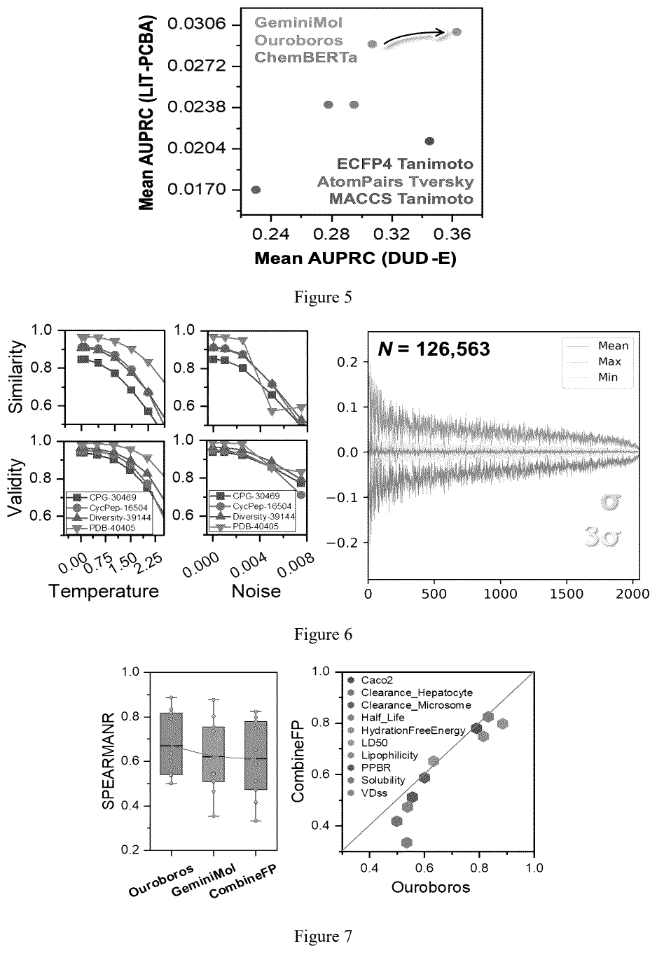

As shown in FIG. 5, the molecular representation base model (i.e., Ouroboros molecular encoder) in this embodiment, after training, demonstrates excellent performance in benchmark tests. The involved benchmark test sets include DUD-E (a manually constructed virtual screening benchmark test set, considered the gold standard test set in the field of virtual screening) and LIT-PCBA (a real-world virtual screening test set derived from high-throughput experimental data). These two test sets are constructed in different ways. During the research, comparisons were made with the PhaseShape method using experimental structures as reference conformations, the GeminiMol method with a similar pre-training scheme, and three traditional molecular fingerprint methods. The results show that the Ouroboros molecular encoder performs best in both benchmark tests. This fully indicates that the molecular representation base model in the present invention has good generalization performance, and its representation ability is stronger compared to traditional molecular fingerprints, especially when processing the LIT-PCBA dataset from real-world data, the advantage is more significant. Among them, the GeminiMol method is a molecular representation method that uses different molecular similarity descriptors for pre-training, but this method does not have a matching molecular generation decoder.

Typically, the Transformer decoder structure used in the present invention should have 16-64 attention heads, a hidden feature dimension between 1024 and 4096 dimensions, and more than 4 network layers. The token set covers all common characters in SMILES. During training, a greedy matching mechanism is used to distinguish elements that are linguistically similar but chemically different, such as C and Cl, B and Br, thereby representing different elements with different tokens.

The training of the molecular decoder can select synthesizable molecules from public datasets or commercial compound libraries (obtainable by purchase). In this embodiment, synthesizable compounds from multiple sources were used, with 128 different molecules used for training per training step. The model reached convergence at the 50,000th training step. During molecular generation, the similarity and validity indicators were used to test the molecular structure decoder's ability to restore the molecular structure corresponding to the molecular encoding and the effectiveness of generating new molecules, respectively. As shown in FIG. 6, during molecular structure decoding, by increasing temperature and applying different levels of noise, the molecular structure decoder in this embodiment was able to generate more new molecules with lower similarity to the original molecule (high diversity) and reasonable structures while maintaining high molecular validity. However, when the temperature exceeded 1.50 (at which point token sampling becomes too aggressive) or the noise exceeded 0.005 (beyond the numerical distribution range of the molecular encoding features themselves), the validity of the molecular decoder decreased.

Given that the encoding and decoding of molecular structures are trained independently, the molecular property prediction model in the present invention uses a one-dimensional molecular vector to predict molecular attributes. Implementation methods include but are not limited to multiple linear regression, decision trees, gradient boosting machines, multilayer perceptions (deep neural networks), etc.

In this embodiment, a set of molecular property prediction models was trained using a multilayer perceptron to predict properties such as water solubility, membrane permeability, clearance rate, half-life, toxicity, and tissue distribution of molecules. The results shown in FIG. 7 indicate that the method of the present invention (labeled as “Ouroboros” in FIG. 7) performs better in property prediction than the GeminiMol model, as well as the combined method of multiple molecular fingerprints (ECFP4, FCFP6, AtomPairs, TopologicalTorsion) CombineFP with automated machine learning (AutoGluon). In terms of the Spearman rank correlation coefficient between predicted values and true values, the Ouroboros prediction model not only performs better in properties where CombineFP performs well, such as water and free energy, lipophilicity, but also improves prediction performance in tasks where CombineFP performs poorly, such as half-life, clearance rate, and tissue distribution.

Since the molecular encoding in the present invention can demonstrate excellent performance in drug virtual screening tasks even independently of the molecular decoder and property predictor, with the help of the Ouroboros framework, optimization of the molecular encoding vector can be achieved and applied to targeted molecular structure migration with pharmacological significance. This function enables the molecular encoding vector to migrate from a starting molecular structure to a target molecular structure, capturing structural changes throughout the migration path, thereby discovering new chemical scaffolds and new molecules with the potential for both scaffold pharmacodynamic characteristics.

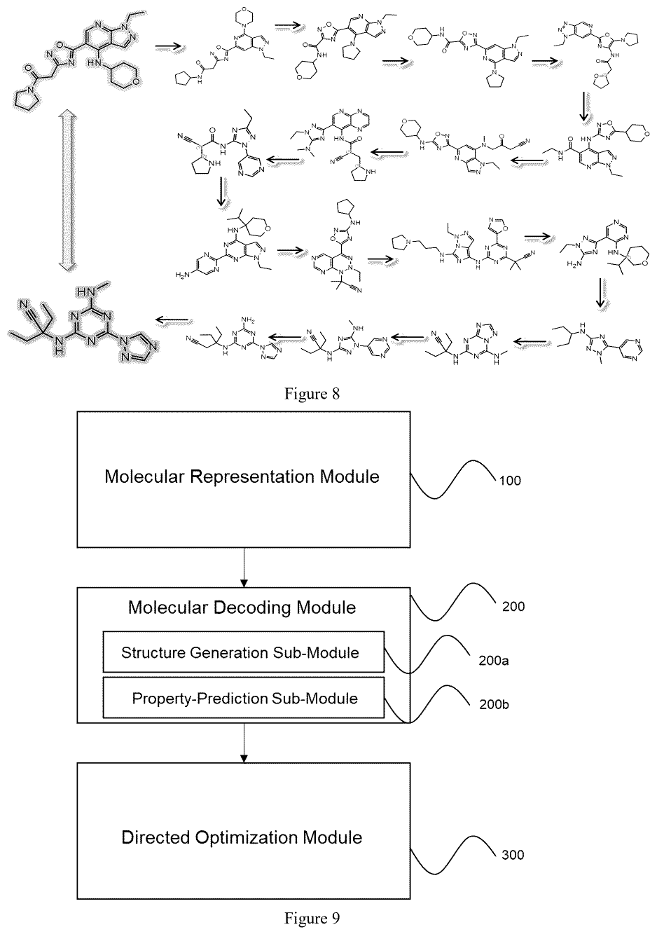

In this embodiment, two PDE4B inhibitors with different scaffolds were selected, and their molecular encoding vectors were optimized using the AdamW optimizer. The loss function was set as the similarity to the molecular encoding vector of the target molecule, and the molecular vector was decoded at each optimization step. As shown in FIG. 8, as the migration process progresses, the molecular structure gradually deviates from the starting structure, undergoes significant scaffold changes, substructures gradually transform towards the target molecule, and a large number of structures similar to the target molecule are generated in the late stages of optimization. During the migration process, other molecular property optimization items can also be added to the loss function, thereby achieving optimization of molecular drug-like properties during the migration process.

### Embodiment 2

Based on the same inventive concept as Embodiment 1, the present invention also provides a drug molecule targeted generation system configured to implement the drug molecule targeted generation method described in Embodiment 1. Referring to FIG. 9, the drug molecule targeted generation system includes: a molecular representation module 100, a decoding module 200, and a molecular targeted optimization module 300.

The molecular representation module 100 is configured to construct a molecular representation base model, and based on the molecular representation base model, perform reading, information updating, information pooling, and information integration of molecular structures, generate a one-dimensional molecular encoding vector capable of representing molecular structure information and pharmacodynamic property information.

The decoding module 200 is configured to construct a molecular structure generation model and a molecular property prediction model, perform segmented training on the molecular structure generation model and the molecular property prediction model based on the one-dimensional molecular encoding vector, to obtain molecular structural formulas or molecular property prediction results.

The molecular targeted optimization module 300 is configured to evaluate the interaction between drug molecules and different targets based on the restored drug molecular structural formulas and molecular property prediction results, and develop multi-target drugs.

Specifically, the decoding module 200 includes a molecular structure generation submodule 200a and a molecular property prediction submodule 200b. The molecular structure generation submodule 200a restores the one-dimensional molecular encoding vector to a molecular structural formula via the molecular structure generation model through dimension expansion, feature transformation, and decoding; the molecular property prediction submodule 200b predicts one or more molecular properties based on the one-dimensional molecular encoding vector via the molecular property prediction model.

This embodiment proposes a drug molecule targeted generation system configured to implement the aforementioned drug molecule targeted generation method. Therefore, the specific implementation methods in the drug molecule targeted generation system can be found in the embodiment section of the aforementioned drug molecule targeted generation method. For example, the molecular representation module 100, decoding module 200, and molecular targeted optimization module 300 are configured to implement steps S1, S2, and S3 in the drug molecule targeted generation method of Embodiment 1, respectively. Therefore, their specific implementation methods can refer to the descriptions of the corresponding embodiments. To avoid redundancy, they are not repeated here.

Those skilled in the art will understand that the embodiments of the present application can be provided as methods, systems, or computer program products. Therefore, the present application can take the form of entirely hardware embodiments, entirely software embodiments, or embodiments combining software and hardware. Moreover, the present application can take the form of a computer program product implemented on one or more computer-usable storage media (including but not limited to disk storage, CD-ROM, optical storage, etc.) containing computer-usable program code.

The present application is described with reference to flowcharts and/or block diagrams of methods, devices (systems), and computer program products according to embodiments of the present application. It will be understood that each flow and/or block of the flowcharts and/or block diagrams, and combinations of flows and/or blocks in the flowcharts and/or block diagrams, can be implemented by computer program instructions. These computer program instructions can be provided to the processor of a general-purpose computer, special-purpose computer, embedded processor, or other programmable data processing device to produce a machine, such that the instructions executed by the processor of the computer or other programmable data processing device produce means for implementing the functions specified in one or more flows of the flowchart and/or one or more blocks of the block diagram. These computer program instructions may also be stored in a computer-readable memory that can direct a computer or other programmable data processing device to operate in a particular manner, such that the instructions stored in the computer-readable memory produce an article of manufacture including instruction means which implement the function specified in one or more flows of the flowchart and/or one or more blocks of the block diagram.

These computer program instructions may also be loaded onto a computer or other programmable data processing device to cause a series of operational steps to be performed on the computer or other programmable device to produce a computer-implemented process such that the instructions which execute on the computer or other programmable device provide steps for implementing the functions specified in one or more flows of the flowchart and/or one or more blocks of the block diagram.

Obviously, the above embodiments are merely examples given for clear description and are not limitations of the implementations. For those of ordinary skill in the art, other different forms of changes or variations can be made based on the above description. It is not necessary nor possible to exhaust all implementations here. The obvious changes or variations derived therefrom are still within the protection scope of the present invention.

1. A drug molecule targeted generation method, comprising steps of:
step S1: acquiring a drug molecular structure to construct a molecular representation base model; based on the molecular representation base model, performing information transformation, information updating, information pooling, and information integration operations on chemical space information of the drug molecular structure, generating a one-dimensional molecular encoding vector capable of representing molecular structure information and pharmacodynamic property information, and optimizing the molecular representation base model based on intermolecular similarity in the one-dimensional molecular encoding vector;
step S2: constructing a molecular structure generation model and a molecular property prediction model, performing segmented training on the molecular structure generation model and the molecular property prediction model based on the one-dimensional molecular encoding vector, obtaining a trained molecular structure generation model and a trained molecular property prediction model, comprising:
restoring the one-dimensional molecular encoding vector to a drug molecular structural formula via the trained molecular structure generation model, to ensure a one-to-one correspondence between the one-dimensional molecular encoding vector and its drug molecular structural formula; and
based on the one-dimensional molecular encoding vector, obtaining one or more molecular property prediction results via the trained molecular property prediction model; and
step S3: based on the molecular property prediction results of the one-dimensional molecular encoding vector, optimizing the one-dimensional molecular encoding vector via backpropagation, and restoring the optimized one-dimensional molecular encoding vector to a molecular structure, to achieve targeted optimization of a drug molecule.

2. The drug molecule targeted generation method according to claim 1, wherein in step S1, the generating the one-dimensional molecular encoding vector capable of representing molecular structure information and pharmacodynamic property information comprising:
S11: converting the drug molecular structure into a SMILES string, and standardizing a format of the SMILES string to obtain a preprocessed SMILES string;
S12: converting the preprocessed SMILES string into a molecular graph, and based on atomic and chemical bond markers in the molecular graph, converting the molecular graph into a homogeneous graph composed of nodes representing atoms and edges representing chemical bonds;
S13: updating node representations and edge representations in the homogeneous graph to obtain an updated molecular graph representation;
S14: inputting each node representation from the updated molecular graph representation into a multilayer perceptron to predict attention weight of each node, and inputting the attention weight of each node into a SoftMax activation function for normalization to obtain a normalized attention weight;
S15: multiplying the normalized attention weights with the corresponding node representations, followed by sum pooling, to obtain the one-dimensional molecular encoding vector.

3. The drug molecule targeted generation method according to claim 2, wherein, the updating the node representations and edge representations in the homogeneous graph comprises:
S131: for initial feature representation of each node, projecting the initial feature representation to a higher-dimensional space via linear transformation to obtain input nodes;
S132: for each edge connecting any two input nodes, iteratively updating a current edge feature by concatenating a feature of any node and a feature of the edge, to obtain an updated edge;
S133: based on the updated edge, for each node, aggregating the features of all connected edges and fusing an aggregation result with the feature of the node to iteratively update a current node feature, to obtain a currently updated node,
S134: for any adjacent node of the currently updated node, performing element-wise multiplication of a feature of the adjacent node and a feature of the updated edge to obtain an integrated feature of the adjacent node, thereby calculating the integrated features of all adjacent nodes of the updated node and aggregating them, and performing element-wise multiplication of the aggregated features of the adjacent nodes with features generated by a self-loop attention mechanism to obtain final updated node features.

4. The drug molecule targeted generation method according to claim 3, the updating the node representations and edge representations in the homogeneous graph further comprises: introducing information such as molecular 3D conformation, conformational dynamics, conformational space, pharmacophore features, and molecular shape to update the feature of each node and the feature of each edge in the homogeneous graph.

5. The drug molecule targeted generation method according to claim 1, wherein the optimizing the molecular representation base model based on intermolecular similarity in the one-dimensional molecular encoding vector comprises:
using a multilayer perceptron as a projection head, inputting any two sets of one-dimensional molecular encoding vectors after concatenation and shape adjustment into the projection head to obtain a predicted similarity matrix between the two sets of molecular encoding vectors;
calculating the similarity between different molecules in the drug molecular structure to obtain a true similarity matrix; and
calculating an error between the predicted similarity matrix and the true similarity matrix, and adjusting parameters of the molecular representation base model via backpropagation based on the error.

6. The drug molecule targeted generation method according to claim 5,
wherein any element in the true similarity matrix represents one or more similarities between different molecules.

7. The drug molecule targeted generation method according to claim 1, wherein the obtaining the trained molecular structure generation model comprises:
performing autoregressive decoding on the one-dimensional molecular encoding vector based on the molecular structure generation model, calculating a predicted probability of each character token representing different molecules at a current position, and constructing a predicted vocabulary based on the predicted probabilities;
segmenting the SMILES string converted from the drug molecular structure into multiple character tokens, and summarizing all character tokens based on their actual position information in an original SMILES string to form a true vocabulary;
calculating an error between corresponding character tokens in the predicted vocabulary and the true vocabulary, and adjusting the parameters of the molecular structure generation model via backpropagation based on the character error until the character error falls within a preset range, to obtain the trained molecular structure generation model.

8. The drug molecule targeted generation method according to claim 1, wherein in step S2, the restoring the one-dimensional molecular encoding vector to a drug molecular structural formula via the trained molecular structure generation model comprises:
using the one-dimensional molecular encoding vector as a memory vector, performing autoregressive decoding on the memory vector via a Transformer decoder structure to obtain a feature vector sequence;
performing character decoding on the feature vector sequence via a multilayer perceptron to obtain predicted scores for each character;
based on the predicted scores, calculating a probability distribution value of each character appearing at the current position, and predicting an output character at the current position based on the probability distribution value, after generating a first character, mapping the first character to a low-dimensional embedding vector and concatenating it with previous feature vectors, and inputting the concatenated vector into the multilayer perceptron again to predict a next character, repeating this prediction process until a generated string meets a specified length, ultimately obtaining a SMILES string composed of multiple characters; and restoring the SMILES string to a drug molecular structural formula.

9. The drug molecule targeted generation method according to claim 1, wherein the obtaining one or more molecular property prediction results via the trained molecular property prediction model comprises transforming the one-dimensional molecular encoding vector into molecular property prediction values via a deep neural network after multiple layers of dimension expansion, batch normalization, and activation functions.

10. A drug molecule targeted generation system, being configured to implement the drug molecule targeted generation method according to claim 1, the drug molecule targeted generation system comprising:
a molecular representation module, configured to acquire a drug molecular structure to construct a molecular representation base model, and based on the molecular representation base model, perform information transformation, information updating, information pooling, and information integration operations on the chemical space information of the drug molecular structure, generate a one-dimensional molecular encoding vector capable of representing molecular structure information and pharmacodynamic property information, and optimize the molecular representation base model based on intermolecular similarity in the one-dimensional molecular encoding vector;
a decoding module, configured to construct a molecular structure generation model and a molecular property prediction model, perform segmented training on the molecular structure generation model and the molecular property prediction model based on the one-dimensional molecular encoding vector, obtain a trained molecular structure generation model and a trained molecular property prediction model, and respectively obtain molecular structural formulas and molecular property prediction results via the trained molecular structure generation model and the trained molecular property prediction model; and
a molecular targeted optimization module, configured to optimize the one-dimensional molecular encoding vector via backpropagation based on the molecular property prediction results of the one-dimensional molecular encoding vector, and restore the optimized one-dimensional molecular encoding vector to a molecular structure, to achieve targeted optimization of a drug molecule;
wherein, the decoding module comprises a molecular structure generation submodule and a molecular property prediction submodule, the molecular structure generation submodule restores the one-dimensional molecular encoding vector to a molecular structural formula via the trained molecular structure generation model; the molecular property prediction submodule obtains one or more molecular property prediction results based on the one-dimensional molecular encoding vector via the trained molecular property prediction model.
